(19) United States
(12) Patent Application Publication
(10) Pub. No.: US 2014/0330396 A1
(43) Pub. Date: Nov. 6, 2014

(54) DEVICE CONTROL SYSTEM BY PHYSIOLOGICAL SIGNALS AND METHOD THEREOF

(71) Applicants: Chih-Tsung Chang, New Taipei City (TW); Hou-Cheng Lu, Taoyuan (TW); Shu-Fen Ko, Miaoli (TW); Mei-Hui Chao, New Taipei City (TW)

(72) Inventors: Chih-Tsung CHANG, New Taipei City (TW); Jyun-Jie SIE, Hsinchu (TW); Hou-Cheng LU, Taoyuan (TW); Shu-Fen KO, Miaoli (TW); Mei-Hui CHAO, New Taipei City (TW)

(73) Assignees: Chih-Tsung Chang, New Taipei City (TW); Hou-Cheng Lu, Taoyuan (TW); Shu-Fen Ko, Miaoli (TW); Mei-Hui Chao, New Taipei City (TW)

(21) Appl. No.: 14/266,823

(22) Filed: Apr. 30, 2014

(30) Foreign Application Priority Data
May 2, 2013 (TW) 102115731

(51) Int. Cl. G05B 15/02; A61B 5/0496; A61B 5/0488; A61B 5/0402; A61B 5/0476

(52) U.S. Cl. CPC G05B 15/02; A61B 5/0402; A61B 5/0476; A61B 5/0488; A61B 5/0496
USPC 700/11

(57) ABSTRACT

A control system and method based on a physiological signal are disclosed, by which a user wears a detection device on the user body to be detected with a physiological signal of the user body, a control server may process the physiological signal to generate a corresponding control command, and thus an under-control device may be controlled to operate based on the control command. By using the technical means, the present invention may achieve a technical efficacy of controlling the under-control device based on the user's physiological signal.

### CROSS-REFERENCE TO RELATED APPLICATION

This present application claims priority to TAIWAN Patent Application Serial Number 102115731, filed on May 2, 2013, which is herein incorporated by reference in its entirety.

### BACKGROUND OF RELATED ART

1. Technical Field

The present invention relates to a device control system and method, and particularly to a device control system and method based on a physiological signal.

2. Related Art

Owing to improvement and development in the technical field, more and more devices are developed to facilitate human's ordinary life, such as lamp, air-conditioner, and fan, with which human beings may lead a comfortable and convenient daily life.

At present, the above devices comprising the lamp, air-conditioner, and fan considerably relies upon a user's manual operations, such as the manual on/off operation and manual setting operation. Once the user may not manually operate the device, such as the case when the user is in sleep, the lamp, air-conditioner, fan, and the similar devices may not be adjusted in settings based on the user's condition, such as the settings of temperature and rotation speed of the air-conditioner and fan. Under such circumstances, the user may be imposed of a burden in addition to an energy waste.

In view of the above issues, a previous time setting function for the lamp, air-conditioner, and fan is provided and thus the on/off control is given a reference, thereby the energy waste and user body's burden issues are soothed. However, when the environment, such as temperature and humidity, changes, the lamp, air-conditioner, and fan may not be changed again, the comfortable and convenient ordinary daily life becomes failed.

In view of the above, it may be known that there has long been the issue encountered in the prior art that a user may not actively control a device and the device may thus not be controlled based on the user's condition

### SUMMARY

In view of the issue encountered in the prior art that a user may not actively control a device and the device may thus not be controlled based on the user's condition, the present invention discloses a device control system and method based on a physiological signal.

According to the present invention, the device control system based on a physiological signal comprises a control server, further comprising a server receiver module, receiving the physiological signal; a signal processing module, processing a signal based on the physiological signal to generate a signal result; a query module, querying from a look-up table a control command corresponding to the signal result; and a server transmitter module, transmitting the control command; a detection device, being worn on a user body, further comprising a detection module, detecting a physiological signal from the user body; and a connection module, establishing a connection with the control server, and providing the physiological signal to the server receiver module; and an under-control device, establishing a connection with the control server, and acquiring the control command from the server transmitter module to perform an operation corresponding thereto based on the control command.

According to the present invention, the device control method based on a physiological signal comprising steps of wearing a detection device on a user body to detect a physiological signal of the user body; establishing a connection between the detection device and the control server, and providing the physiological signal to the control server from the detection device; processing the physiological signal by the control server based on the physiological signal to generate a signal result; querying a control command corresponding to the signal result queried from a look-up table by the control server; and establishing a connection between an under-control device and the control server, and acquiring the control command from the control server, and performing an operation based on the control command.

As compared to the prior art, the system and method of the present invention has the difference that the user wears the detection device on the user body to be detected with a physiological signal of the user body, the control server may process the physiological signal to generate the corresponding control command, and thus the under-control device may be controlled to operate based on the control command.

By using the technical means, the present invention may achieve the technical efficacy of controlling the under-control device based on the user's physiological signal.

### BRIEF DESCRIPTION OF THE DRAWINGS

The present invention will be better understood from the following detailed descriptions of the preferred embodiments according to the present invention, taken in conjunction with the accompanying drawings, in which:

FIG. 1 depicts a systematic block diagram of a device control device based on a physiological signal according to the present invention;

FIG. 2 depicts a flowchart diagram of a device control method based on a physiological signal according to the present invention;

FIG. 3 depicts a systematic architecture of the device control device based on a physiological signal according to a first embodiment of the present invention;

FIG. 4 depicts a schematic diagram of a physiological signal used in the device control device based on the physiological signal according to a first embodiment of the present invention;

FIG. 5 depicts a schematic diagram of a look-up table used in the device control device based on a physiological signal according to a first embodiment of the present invention;

FIG. 6 depicts a systematic architecture of the device control device based on a physiological signal according to a second embodiment of the present invention;

FIG. 7 depicts a schematic diagram of the physiological signal used in the device control device based on a physiological signal according to a second embodiment of the present invention;

FIG. 8 depicts a schematic diagram of a brain wave signal used in the device control device based on a physiological signal according to a second embodiment of the present invention; and;

FIG. 9 depicts a schematic diagram of the look-up table used in the device control device based on a physiological signal according to a second embodiment of the present invention.

### DETAILED DESCRIPTION

The present invention will be apparent from the following detailed description, which proceeds with reference to the accompanying drawings, wherein the same references relate to the same elements.

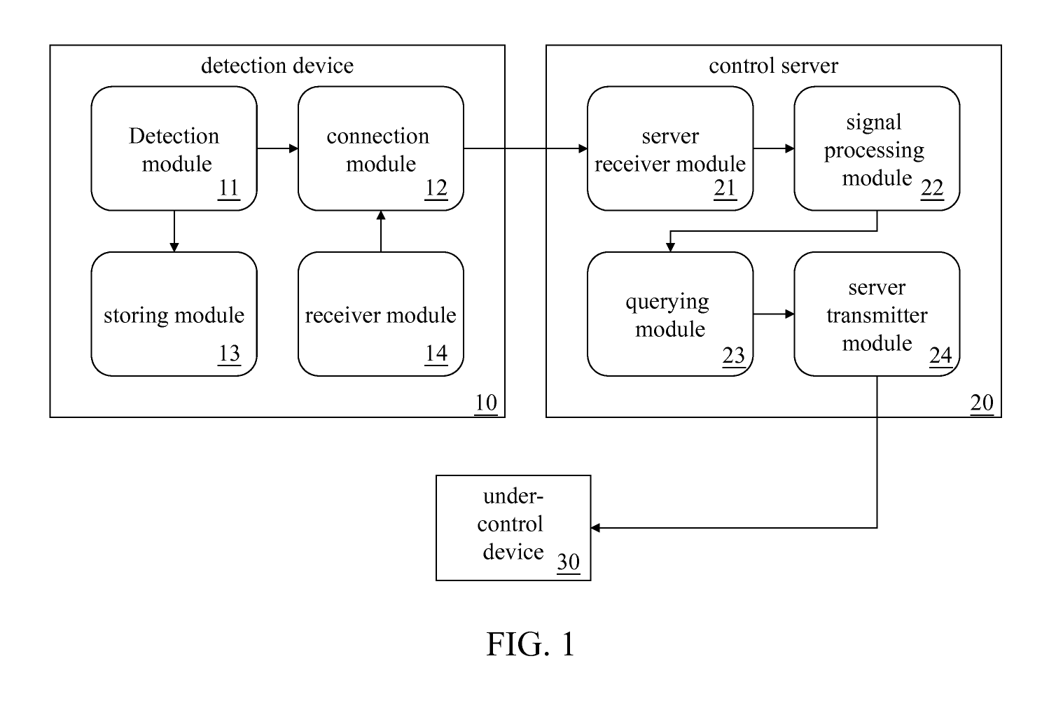

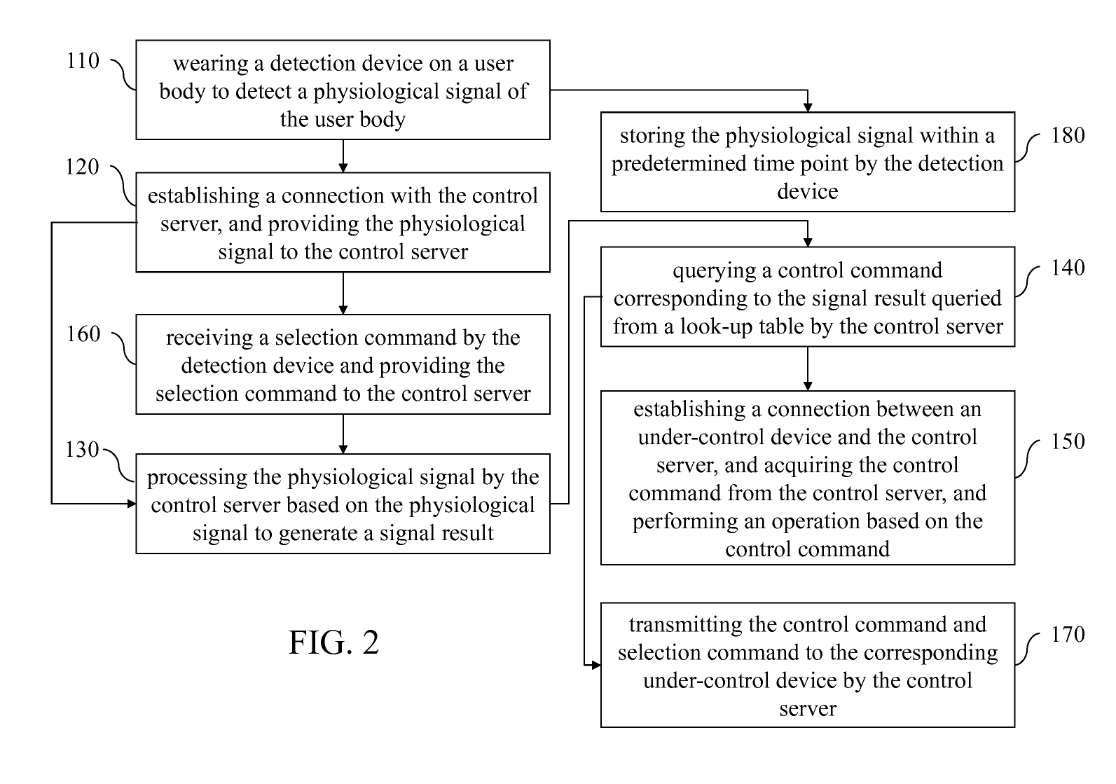

In what follows, how a device is controlled based on a physiological signal according to a first embodiment of the present invention is explained with reference to FIG. 1 and FIG. 2, in which FIG. 1 depicts a systematic block diagram of a device control device based on a physiological signal according to the present invention, and FIG. 2 depicts a flowchart diagram of a device control method based on a physiological signal according to the present invention.

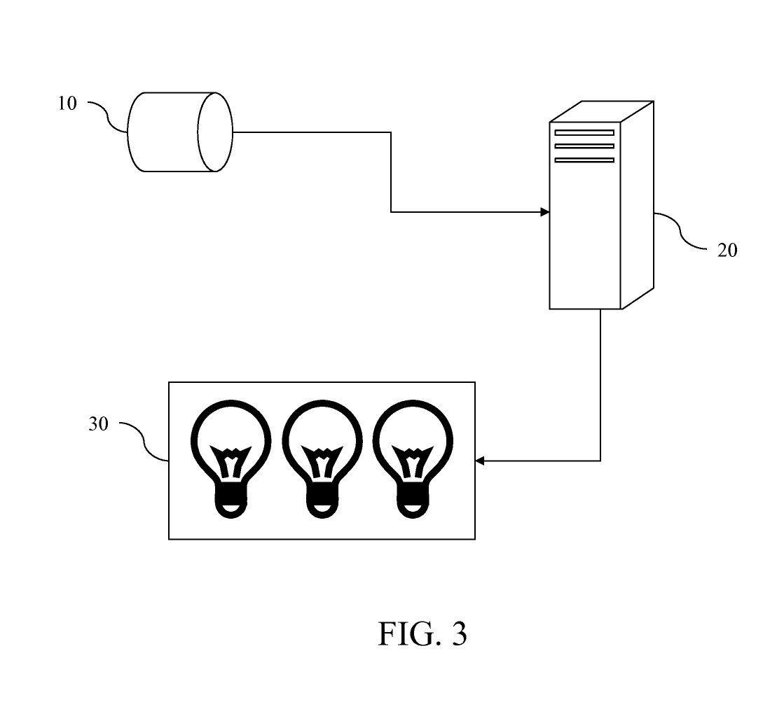

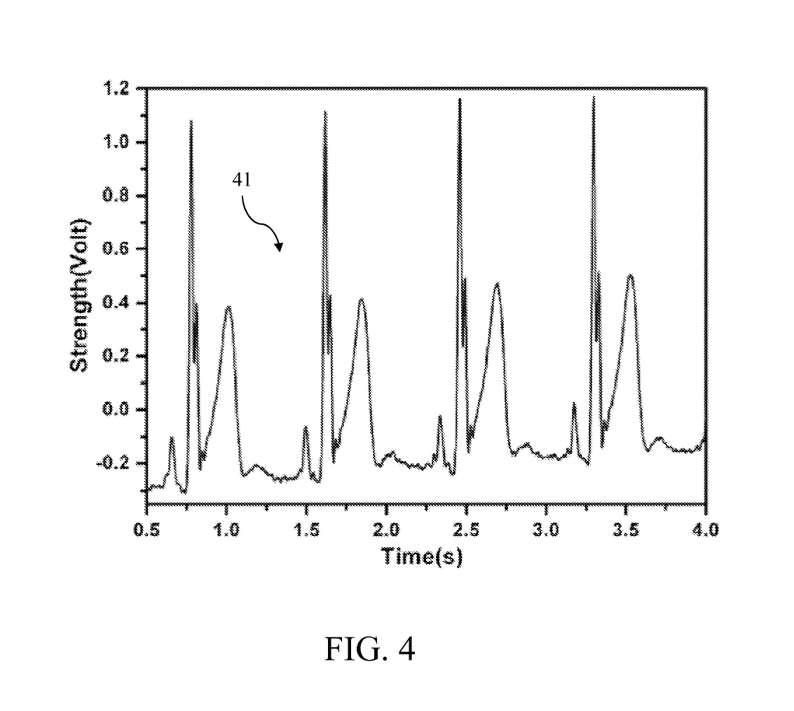

Referring simultaneously to FIG. 3 and FIG. 4, FIG. 3 depicts a systematic architecture of the device control device based on a physiological signal according to a first embodiment of the present invention, and FIG. 4 depicts a schematic diagram of a physiological signal used in the device control device based on a physiological signal according to a first embodiment of the present invention.

In the first embodiment, a detection device 10 is presented as a wrist ring. That is the detection device 10 is worn on a wriest of a user. The detection device 10 detects a physiology signal 41 of the user through an infrared detection or a dry electrode detection fashions (S110). The detected physiological signal 41 in the first embodiment is a cardiograph signal 41, and which is schematically shown in FIG. 4. This is merely an example, without limiting the present invention.

In the detection device 10, the detection module 11 detects the physiological signal 41 in a predetermined time point, and a storing module 13, an unnecessary module, is further comprised to store the physiological signal in a predetermined time point (S180). The step S180 is not a necessary step, and the predetermined time may be 1 hr., 12 hr., 24 hr., and the like. Furthermore, the storing module 13 of the detection device 10 may be stored through an external storage device, such as a SD card series, a SIM, and the like card. These are merely examples, without limiting the present invention.

Thereafter, the detection device 10 may be connected with the control server 20 in a wireless manner through the connection module 12, wherein the wireless transmission manner may comprises Bluetooth, Wi-Fi, wireless network, etc. These are merely examples, without limiting the present invention. After the detection device 10 and the control server 20 are wirelessly connected through the connection module 12, the connection module 12 of the detection device 10 may provide the detected physiological signal 41 by the detection module 11 to the control server 20 (S120).

The server receiver module 21 of the control server 20 may at this time acquire the physiological signal 41 from the connection module 12 of the detection device 10. After the physiological signal 41 is acquired at the server receiver module 21 of the control server 20 from the connection module 12 of the detection device 10, a signal processing module 22 at the control server 20 may process the physiological signal 41 and thus generates a signal result based on the processed physiological signal (S130).

At the control server 20, the signal processing module 22 analyzes and computes a heart rate change by using a heart rate variability (HRV) on a continuous heart rate interval 200 to 500, which is merely an example without limiting the present invention, by which a discrete Fourier transform method is used to transform a temporal heart rate series into a frequency domain, by which the heart rate change may be presented in a power spectral density (PSD) or a spectral distribution form. In this manner, the physiological signal 41 may be computed to obtain an average frequency value. Typically, heart rate intervals have their frequency lower than 1 Hz, and two main portions: a higher frequency area and a lower frequency. Generally, the higher frequency area reflects an activity of parasympathetic nerve, while the lower frequency area is related to adjustment and control of concurrent sympathetic nerve. In the first embodiment, the signal processing module 22 at the control server 20 outputs its processed signal as “0.30 Hz”.

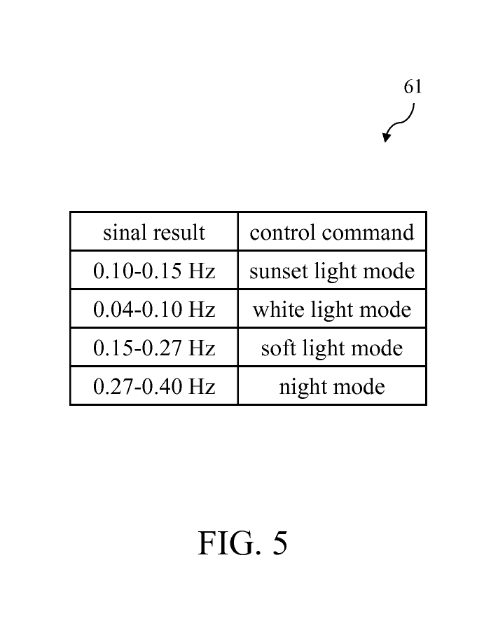

Subsequently, referring simultaneously to FIG. 3 and FIG. 5, in which FIG. 5 depicts a schematic diagram of a look-up table used in the device control device based on a physiological signal according to a first embodiment of the present invention.

At the control server 20, the querying module 23 may obtain a control command “night mode” from a look-up table 61 by querying the signal result “0.30 Hz” (S140). In response, the server transmitter module 24 at the control server 20 provides the control command “night mode” to the under-control device 30.

In this first embodiment, the under-control device 30 is exemplified by a lamp, an air-conditioner, a fan, and the like, which are merely examples without limiting the present invention. Between the under-control device 30 and the control server 20, a wireless transmission manner is used for a connection, which may include Bluetooth, Wi-Fi, and wireless network, and the like, and which are merely examples without limiting the present invention.

When the under-control device 30 receives the control command as “night mode” from the server transmitter module 24 at the control server 20, the under-control device 30 may perform a corresponding operation based on the control command “night mode”. That is, the under-control device 30 may switch an illumination mode of an LED lamp based on the control command “night mode” (S150). The control command “night mode” refers to an action switch the LED lamp into a red LED as a main illumination, along with few warm white LED, green LED and blue LED as a complemented color, by which a night illumination may be simulated. When the control command is “soft light mode”, the control command refers to an action switching the LED lamp into the warm white LED as the main illumination, along with red LED, green LED, and blue LED as a complemented color, lemon yellow, to simulate a sunset illumination. When the control command is “sunrise light mode”, the control command refers to an action switching the LED lamp into the warm white LED as the main illumination, along with red LED, green LED, and blue LED as a complemented color, sky blue, to simulate a sunset illumination.

As such, different illumination effects may be presented based on different users' physiological signals, i.e. the under-control device 30 may be controlled based on the different physiological signal of the user.

In addition, the detection device 10 further comprises a receiver module 14, which is not a necessary module, and used to receive a selection command inputted by the user, wherein the selection command relates to a selection of different under-control devices 30. And, the selection command is provided to the server receiver module 21 at the control server 20 through the connection module 12 of the detection device 10 (S160). However, step S160 is not a necessary step. At this time, the server transmitter module 24 at the control server 20 may transmit the control command to the under-control device 30 corresponding to the selection command (S170). However, this step is not a necessary step. In this manner, a single detection device 10 may be used to provide a control result on different under-control devices 30.

Thereafter, a second embodiment for the control on a device based on a physiological signal according to the present invention will be explained as follows, with reference to FIG. 1 and FIG. 2.

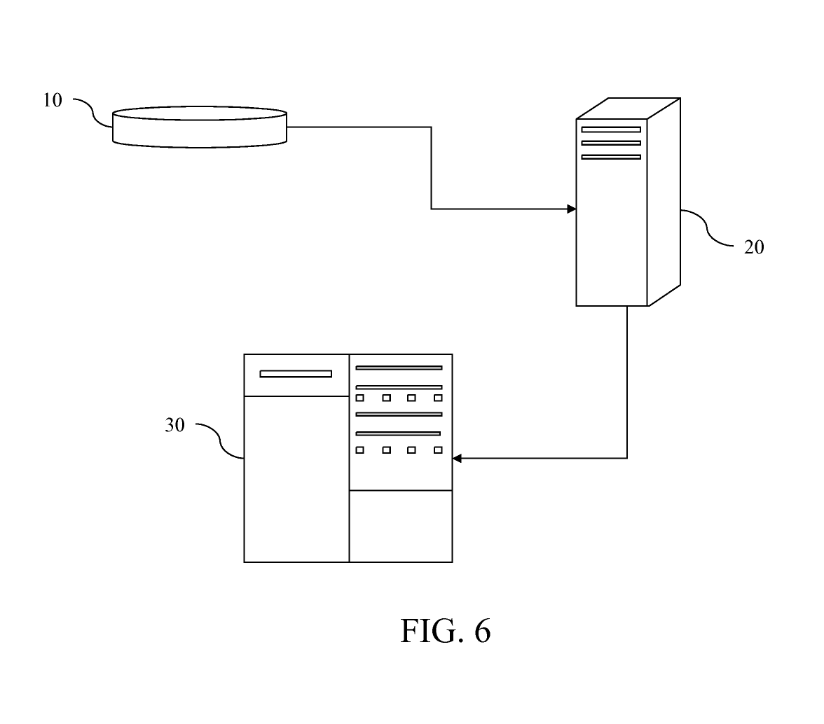

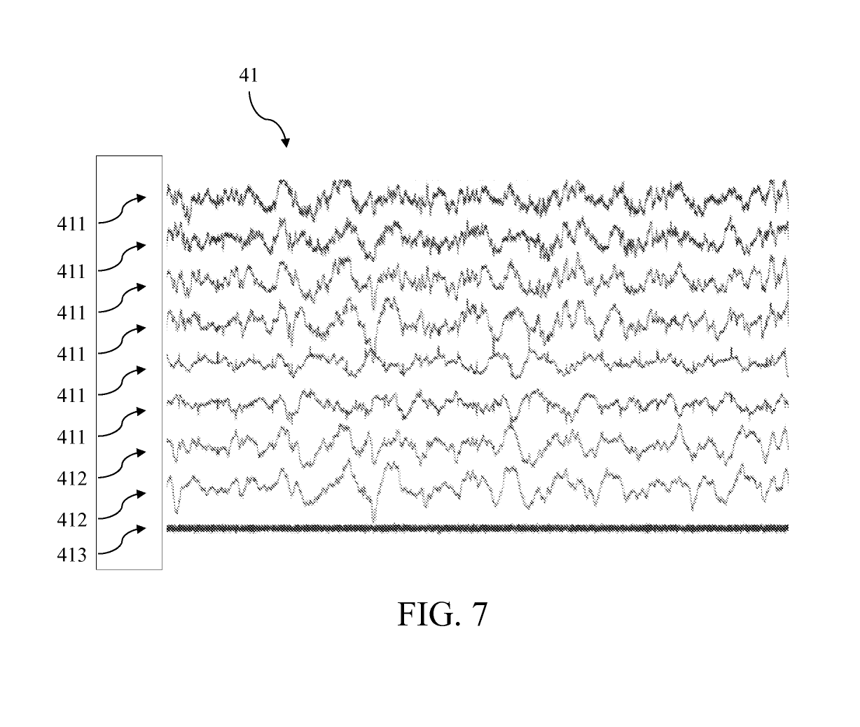

Thereafter, referring simultaneously reference to FIG. 6 and FIG. 7, in which FIG. 6 depicts a systematic architecture of the device control device based on a physiological signal according to a second embodiment of the present invention, while FIG. 7 depicts a schematic diagram of the physiological signal used in the device control device based on a physiological signal according to a second embodiment of the present invention.

In the second embodiment, a detection device 10 is presented as a head ring. That is, the detection device 10 is worn on a head of a user. The detection device 10 detects a physiology signal 41 of the user through a brain wave detection fashion (S110). The detected physiological signal 41 in the second embodiment is a brain wave signal, and which is schematically shown in FIG. 7. In FIG. 7, sex brain electroencephalograms (EEGs) 411, two electroculograms (EOGs) 412, and an electroencephalogram (EMG) 413 are schematically shown. This is merely an example, without limiting the present invention.

In the detection device 10, the detection module 11 detects the physiological signal 41 in a predetermined time point, and a storing module 13, an unnecessary module, is further comprised to store the physiological signal in a predetermined time point (S180). The step S180 is not a necessary step, and the predetermined time may be 1 hr., 12 hr., 24 hr., and the like. Furthermore, the storing module 13 of the detection device 10 may be stored through an external storage device, such as a SD card series, a SIM, and the like card. These are merely examples, without limiting the present invention.

Thereafter, the detection device 10 may be connected with the control server 20 in a wireless manner through the connection module 12, wherein the wireless transmission manner may comprises Bluetooth, Wi-Fi, wireless network, etc. These are merely examples, without limiting the present invention. After the detection device 10 and the control server 20 are wirelessly connected through the connection module 12, the connection module 12 of the detection device 10 may provide the detected physiological signal 41 by the detection module 11 to the control server 20 (S120).

The server receiver module 21 at the control server 20 may at this time acquire the physiological signal 41 from the connection module 12 of the detection device 10. After the physiological signal 41 is acquired at the server receiver module 21 of the control server 20 from the connection module 12 of the detection device 10, a signal processing module 22 at the control server 20 may process the physiological signal 41 and thus generates a signal result based on the processed physiological signal (S130).

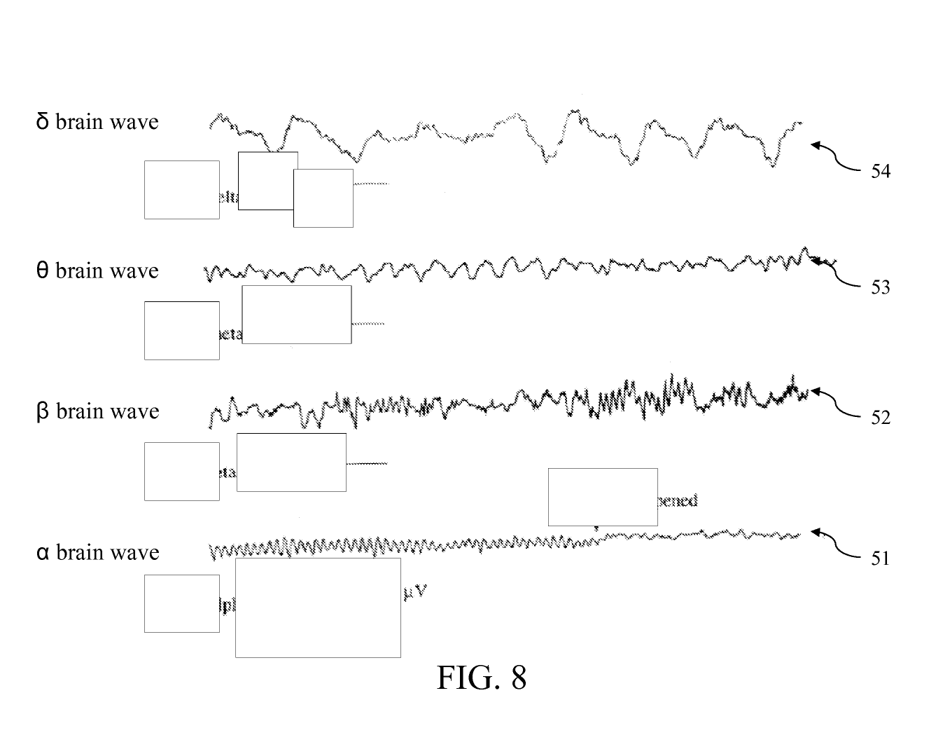

Thereafter, referring simultaneously to FIG. 6, FIG. 7 and FIG. 8, in which FIG. 8 depicts a schematic diagram of a brain wave signal used in the device control device based on a physiological signal according to a second embodiment of the present invention.

At the control server 20, the signal processing module 22 determines a sleep state of the user based on the six EEGs 411, two EOGs 412 and one EMG 413, and the determination rules are explained as what follows.

When the brain wave EEGs 411 are obtained based mainly on a β brain wave 52, with concurrently EEGs 412 having bigger amplitudes and EMG 413 having bigger amplitudes, the signal processing module 22 at the control server 20 may determine the physiological signal 41 as having a signal result “awakened state”. The β brain wave schematically shown in FIG. 8 is merely an example, without limiting the present invention.

When the brain wave EEGs 411 are obtained based mainly on an a brain wave 51, with concurrently EEGs 412 having gentle waves and keen waves and smaller amplitudes and EMG 413 having smaller amplitudes, the signal processing module 22 at the control server 20 may determine the physiological signal 41 as having a signal result “first stage sleep state”. The α brain wave schematically shown in FIG. 8 is merely an example, without limiting the present invention.

When the brain wave EEGs 411 are obtained based mainly on a θ brain wave 53, with concurrently EEGs 412 having spindle or L-composite waves and smaller amplitudes and being regularly presented, and EMG 413 having smaller amplitudes, the signal processing module 22 at the control server 20 may determine the physiological signal 41 as having a signal result “second stage sleep state”. The α brain wave schematically shown in FIG. 8 is merely an example, without limiting the present invention.

When the brain wave EEGs 411 are obtained based mainly on a δ brain wave 54, with concurrently EEGs 412 having above 20% of high-amplitude slow waves and smaller amplitudes representing the high-amplitude slow wave, and EMG 413 having smaller amplitudes, the signal processing module 22 at the control server 20 may determine the physiological signal 41 as having a signal result “third stage sleep state”. The δ brain wave schematically shown in FIG. 8 is merely an example, without limiting the present invention.

The above signal results includes “awakened state”, “first stage sleep stage”, “second sleep state”, “third sleep state”, and the like, which are merely examples without limiting the present invention. With reference to the sleep states determination rules, the physiological signal 41 having the six EEGs, 411 two EEGs 412, and one EMGs 413 is processed at the signal processing module 22 of the control server 20, and thus a signal result “third stage sleep state” is obtained from the processed physiological signal.

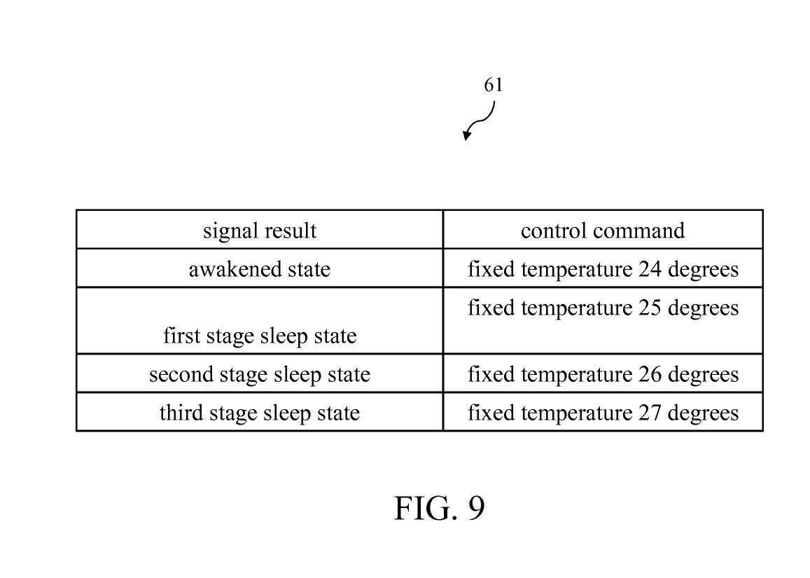

Subsequently, referring simultaneously to FIG. 6 and FIG. 9, in which FIG. 9 depicts a schematic diagram of the look-up table used in the device control device based on a physiological signal according to a second embodiment of the present invention.

At the control server 20, the querying module 23 may obtain a control command “night mode” from a look-up table 61 by querying the signal result “0.30 Hz” (S140). In response, the server transmitter module 24 at the control server 20 provides the control command “night mode” to the under-control device 30.

FIG. 9 depicts a schematic diagram of a look-up table used in the device control device based on a physiological signal according to a second embodiment of the present invention.

At the control server 20, the querying module 23 may obtain a control command “the third stage sleep state” from a look-up table 61 by querying the signal result “fixed temperature 27 degrees” (S140). In response, the server transmitter module 24 at the control server 20 provides the control command “fixed temperature 27 degrees” to the under-control device 30 (S140).

In this second embodiment, the under-control device 30 is exemplified by an air-conditioner, which is merely an example without limiting the present invention. Between the under-control device 30 and the control server 20, a wireless transmission manner is used for a connection, which may include Bluetooth, Wi-Fi, and wireless network, and the like, and which are merely examples without limiting the present invention.

When the under-control device 30 receives the control command as “fixed temperature 27 degrees” from the server transmitter module 24 at the control server 20, the under-control device 30 may perform a corresponding operation based on the control command “fixed temperature 27 degrees” (S150). That is, the under-control device 30 may set the temperature of the air-conditioner as the “fixed temperature 27 degrees” based on the control command. As a comparative example, “fixed temperature 26 degrees” represents setting the air-conditioner to have a temperature of 26 degrees.

In this manner, different air-conditioner temperature may be achieved based on different physiological signals from the user, i.e. the under-control device 30 may be controlled based on the different physiological signal of the user.

In addition, the detection device 10 further comprises a receiver module 14, which is not a necessary module, and used to receive a selection command inputted by the user, wherein the selection command relates to a selection of different under-control devices 30. And, the selection command is provided to the server receiver module 21 at the control server 20 through the connection module 12 of the detection device 10 (S160). However, the step S160 is not a necessary step. At this time, the server transmitter module 24 at the control server 20 may transmit the control command to the under-control device 30 corresponding to the selection command (S170). However, this step is not a necessary step. In this manner, a single detection device 10 may be used to provide a control result on different under-control devices 30.

In view of the above, the system and method of the present invention has the difference that the user wears the detection device on the user body to be detected with a physiological signal of the user body, the control server may process the physiological signal to generate the corresponding control command, and thus the under-control device may be controlled to operate based on the control command. By using the technical means, the present invention may overcome the issue which the device may not be controlled based on the user's condition when the user may not actively control the device, and further achieve the efficacy of controlling the under-control device based on the user's physiological signal.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A device control system based on a physiological signal, comprising:
a control server, further comprising:
a server receiver module, receiving the physiological signal;
a signal processing module, processing a signal based on the physiological signal to generate a signal result;
a query module, querying from a look-up table a control command corresponding to the signal result; and
a server transmitter module, transmitting the control command;
a detection device, being worn on a user body, further comprising:
a detection module, detecting a physiological signal from the user body; and
a connection module, establishing a connection with the control server, and providing the physiological signal to the server receiver module; and
an under-control device, establishing a connection with the control server, and acquiring the control command from the server transmitter module to perform an operation corresponding thereto based on the control command.

2. The device control system as claimed in claim 1, wherein the detection device is worn on a wrist of the user body, and the physiological signal detected by the detection module is a cardiograph signal, or the detection device is worn on a head portion of the user body, and the physiological signal detected by the detection module is a brain wave signal.

3. The device control system as claimed in claim 2, wherein when the physiological signal is the cardiograph signal, the signal processing module computes an average frequency value of the cardiograph signal to generate the signal result.

4. The device control system as claimed in claim 2, wherein when the physiological signal is the brain wave signal, the signal processing module determines a corresponding sleep stage based on an electroencephalogram (EEG), an electromyogram (EMG) and an electroculogram (EOG), to generate the signal result.

5. The device control system as claimed in claim 1, wherein the connection module and the control server establish the connection in a wireless transmission manner, the wireless transmission manner including Bluetooth, Wi-Fi, and wireless network.

6. The device control system as claimed in claim 1, wherein the under-control module and the control server establish the connection in a wireless transmission manner, the wireless transmission manner including Bluetooth, Wi-Fi, and wireless network.

7. The device control system as claimed in claim 1, wherein the under-control device includes a traditional lamp, an LED lamp, an air conditioner, and a fan.

8. The device control system as claimed in claim 1, wherein the detection device further comprises a receiver module for receiving a selection command, the connection module provides the selection command to the server receiver module, and the server transmitter module transmits the control command to the under-control corresponding to the selection command.

9. The device control system as claimed in claim 1, wherein the detection device further comprises a storing module for storing the physiological signal within a predetermined time point.

10. A device control method based on a physiological signal, comprising steps of:
wearing a detection device on a user body to detect a physiological signal of the user body;
establishing a connection with the control server, and providing the physiological signal to the control server;
processing the physiological signal by the control server based on the physiological signal to generate a signal result;
querying a control command corresponding to the signal result queried from a look-up table by the control server; and
establishing a connection between an under-control device and the control server, and acquiring the control command from the control server, and performing an operation based on the control command.

11. The device control method as claimed in claim 10, wherein the detection device is worn on a wrist of the user body, and the physiological signal detected by the detection module is a cardiograph signal, or the detection device is worn on a head portion of the user body, and the physiological signal detected by the detection module is a brain wave signal.

12. The device control method as claimed in claim 11, wherein when the physiological signal is the cardiograph signal, the signal processing module computes an average frequency value of the cardiograph signal to generate the signal result.

13. The device control method as claimed in claim 11, wherein when the physiological signal is the brain wave signal, the signal processing module determines a corresponding sleep stage based on an electroencephalogram (EEG), an electromyogram (EMG) and an electroculogram (EOG), to generate the signal result.

14. The device control method as claimed in claim 10, wherein the connection module and the control server establish the connection in a wireless transmission manner, the wireless transmission manner including Bluetooth, Wi-Fi, and wireless network.

15. The device control method as claimed in claim 10, wherein the under-control module and the control server establish the connection in a wireless transmission manner, the wireless transmission manner including Bluetooth, Wi-Fi, and wireless network.

16. The device control method as claimed in claim 10, wherein the under-control device includes a traditional lamp, an LED lamp, an air conditioner, and a fan.

17. The device control method as claimed in claim 10, further comprising steps of:
receiving a selection command by the detection device; and
transmitting the control command to the under-control corresponding to the selection command by the control server.

18. The device control method as claimed in claim 10, wherein the detection device further comprises a storing module for storing the physiological signal within a predetermined time point.
